(19) United States
(12) Patent Application Publication
(10) Pub. No.: US 2019/0076384 A1
(43) Pub. Date: Mar. 14, 2019

(54) METHODS FOR TREATING UREA CYCLE DISORDERS

(71) Applicant: Horizon Therapeutics, LLC, Lake Forest, IL (US)

(72) Inventors: Bruce SCHARSCHMIDT, San Francisco, CA (US); Masoud MOKHTARANI, Walnut Creek, CA (US)

(21) Appl. No.: 16/057,335

(22) Filed: Aug. 7, 2018

Related U.S. Application Data
(62) Division of application No. 15/816,711, filed on Nov. 17, 2017.
(60) Provisional application No. 62/555,849, filed on Sep. 8, 2017.

(51) Int. Cl. A61K 31/216; A61K 31/5517; A61K 45/06; A61P 3/00

(52) U.S. Cl. CPC A61K 31/216; A61K 31/5517; A61P 3/00; A61K 45/06

(57) ABSTRACT

Provided are methods of administering glycerol phenylbutyrate to a patient in need thereof, wherein said patient is also being treated with a CYP3A4 substrate having a narrow therapeutic index, midazolam or a pharmaceutically acceptable salt thereof, or celecoxib.

This application claims the benefit of U.S. Provisional Application, 62/555,849, filed Sep. 8, 2017, which is incorporated herein by reference for all purposes.

Urea cycle disorders (UCD) are inborn errors of metabolism caused by a deficiency in one of six enzymes or two mitochondrial transport proteins involved in the production of urea, resulting in accumulation of toxic levels of ammonia in the blood (hyperammonemia). UCD subtypes include those caused by an X-linked mutation and corresponding deficiency in ornithine transcarbamylase (OTC) and those caused by autosomal recessive mutations with corresponding deficiencies in argininosuccinate synthetase (ASS), carbamyl phosphate synthetase (CPS), argininosuccinate lyase (ASL), arginase (ARG), N-acetylglutamate synthetase (NAGS), ornithine translocase (HHH), and aspartate glutamate transporter (CITRIN). These are rare diseases, with an overall estimated incidence in the United States of approximately 1 in every 35,000 live births. UCD is suspected when a subject experiences a hyperammonemic event with an ammonia level >100 μmol/L accompanied by signs and symptoms compatible with hyperammonemia in the absence of other obvious causes and generally confirmed by genetic testing.

The severity and timing of UCD presentation vary according to the severity of the deficiency, which may range from minor to extreme depending on the specific enzyme or transporter deficiency, and the specific mutation in the relevant gene. UCD patients may present in the early neonatal period with a catastrophic illness, or at any point in childhood, or even adulthood, after a precipitating event such as infection, trauma, surgery, pregnancy/delivery, or change in diet. Acute hyperammonemic episodes at any age carry the risk of encephalopathy and resulting neurologic damage, sometimes fatal, but even chronic, sub-critical hyperammonemia can result in impaired cognition. UCDs are therefore associated with a significant incidence of neurological abnormalities and intellectual and developmental disabilities over all ages. UCD patients with neonatal-onset disease are especially likely to suffer cognitive impairment and death compared with patients who present later in life.

Management of acute hyperammonemic crises may require hemodialysis and/or intravenous (IV) administration of sodium phenylacetate (NaPAA) and sodium benzoate (NaBz) (the admixture is marketed in the U.S. as AMMONUL®). Orthotopic liver transplantation may also be considered for patients with severe disease that manifests itself in the neonatal period. Long-term UCD management is directed toward prevention of hyperammonemia and includes restriction of dietary protein; arginine and citrulline supplementation, which can enhance waste nitrogen excretion for certain UCDs; and oral, ammonia-scavenging drug therapy that provides an alternate path for waste nitrogen removal (RAVICTI® (glycerol phenylbutyrate, GPB) Oral Liquid or sodium phenylbutyrate (NaPBA; marketed in the U.S. as BUPHENYL® and in the European Union (EU) as AMMONAPS®)).

RAVICTI®, formerly HPN-100, a prodrug of PBA and a pre-prodrug of the active compound phenylacetate (PAA), has been approved in the U.S. for use as a nitrogen-binding agent for chronic management of adults and patients 2 months of age and older with UCDs who cannot be managed by dietary protein restriction and/or amino acid supplementation alone. RAVICTI® is glycerol phenylbutyrate, a triglyceride containing 3 molecules of PBA linked to a glycerol backbone, the chemical name of which is benzenebutanoic acid, 1′,1″-(1,2,3-propanetriyl) ester.

Glycerol phenylbutyrate is used with dietary protein restriction and, in some cases, dietary supplements (e.g., essential amino acids, arginine, citrulline, protein-free calorie supplements). RAVICTI® is not indicated for the treatment of acute hyperammonemia in patients with UCDs, and the safety and efficacy of RAVICTI® for the treatment of NAGS deficiency has not been established. The RAVICTI® Package Insert states the drug is contraindicated in patients less than 2 months of age, stating that children less than 2 months of age may have immature pancreatic exocrine function, which could impair hydrolysis of RAVICTI®, leading to impaired absorption of phenylbutyrate and hyperammonemia; and in patients with known hypersensitivity to phenylbutyrate (signs include wheezing, dyspnea, coughing, hypotension, flushing, nausea, and rash). Pancreatic lipases may be necessary for intestinal hydrolysis of RAVICTI®, allowing release of phenylbutyrate and subsequent formation of PAA, the active moiety. It is not known whether pancreatic and extrapancreatic lipases are sufficient for hydrolysis of RAVICTI®.

The cytochrome P450 enzyme system (CYP450) is responsible for the biotransformation of drugs from active substances to inactive metabolites that can be excreted from the body. In addition, the metabolism of certain drugs by CYP450 can alter their PK profile and result in sub-therapeutic plasma levels of those drugs over time.

There are more than 1500 known P450 sequences which are grouped into families and subfamily. The cytochrome P450 gene superfamily is composed of at least 207 genes that have been named based on the evolutionary relationships of the cytochromes P450. For this nomenclature system, the sequences of all of the cytochrome P450 genes are compared, and those cytochromes P450 that share at least 40% identity are defined as a family (designated by CYP followed by a Roman or Arabic numeral, e.g., CYP3), and further divided into subfamilies (designated by a capital letter, e.g., CYP3A), which are comprised of those forms that are at least 55% related by their deduced amino acid sequences. Finally, the gene for each individual form of cytochrome P450 is assigned an Arabic number (e.g., CYP3A4).

CYP3A isoenzyme is a member of the cytochrome P450 superfamily which constitutes up to 60% of the total human liver microsomal cytochrome P450 and has been found in alimentary passage of stomach and intestines and livers. CYP3A has also been found in kidney epithelial cells, jejunal mucosa, and the lungs. CYP3A is one of the most abundant subfamilies in cytochrome P450 superfamily.

At least five (5) forms of CYPs are found in human CYP3A subfamily, and these forms are responsible for the metabolism of a large number of structurally diverse drugs. In non-induced individuals, CYP3A may constitute 15% of the P450 enzymes in the liver; in enterocytes, members of the CYP3A subfamily constitute greater than 70% of the CYP-containing enzymes.

CYP3A is responsible for metabolism of a large number of drugs including nifedipine, macrofide antibiotics including erythromycin and troleandomycin, cyclosporin, FK506, teffenadine, tamoxifen, lidocaine, midazolam, triazolam, dapsone, diltiazem, lovastatin, quinidine, ethylestradiol, testosterone, and alfentanil. CYP3A is involved in erythromycin N-demethylation, cyclosporine oxidation, nifedipine oxidation, midazolam hydroxylation, testosterone 6-β-hydroxylation, and cortisol 6-β-hydroxylation. CYP3A has also been shown to be involved in both bioactivation and detoxication pathways for several carcinogens in vitro.

CYP2C9 is a cytochrome P450 enzyme with a major role in the oxidation of both xenobiotic and endogenous compounds. CYP2C9, which catalyzes the metabolism of a number of commonly used active agents, including that of warfarin and phenytoin, is also polymorphic. The two most common CYP2C9 allelic variants have reduced activity (5-12%) compared to the wild-type enzyme. Genetic polymorphism also occurs in CYP2C19, for which at least 8 allelic variants have been identified that result in catalytically inactive protein. About 3% of Caucasians are poor metabolizers of active agents metabolized by CYP2C19, while 13-23% of Asians are poor metabolizers of active agents metabolized by CYP2C19.

The Food and Drug Administration requested in vivo drug interaction study for RAVICTI with a CYP3A4/5 substrate based on the following:
The wide range of drugs that are metabolized by CYP3A4;
The significant contribution of CYP3A4 to metabolism in the intestine;
Phenylacetate, which is converted from phenylbutyrate, showed an inhibitory effect on CYP3A4 at a concentration higher than the observed plasma concentrations. Because those potential effects on phenylacetate, the metabolite of RAVICTI, on CYP3A4 could not be ruled.

Phenylacetate also showed an inhibitory effect on CYP2C9 at a concentration higher than the observed plasma concentrations. Again, because of that potential effect, an in vivo drug interaction study was undertaken.

There is a significant, unmet need for methods for administering a nitrogen scavenging drug, such as glycerol phenylbutyrate, to a patient in need thereof, wherein the patient is also being treated with another substance which may interact with the nitrogen scavenging drug. The present disclosure meets these needs.

### SUMMARY

Provided are methods of administering glycerol phenylbutyrate to a patient in need thereof, wherein said patient is also being treated with a CYP3A4 substrate having a narrow therapeutic index, midazolam or a pharmaceutically acceptable salt thereof, or celecoxib.

Also provided is a method of administering glycerol phenylbutyrate to a patient in need thereof, wherein said patient is also being treated with a CYP3A4 substrate having a narrow therapeutic index, comprising administering to the patient a therapeutically effective amount of the glycerol phenylbutyrate, and monitoring the therapeutic effect of the CYP3A4 substrate.

Also provided is a method of administering glycerol phenylbutyrate to a patient in need thereof, wherein said patient is also being treated with celecoxib, comprising administering to the patient a therapeutically effective amount of the glycerol phenylbutyrate.

Also provided is a method of administering glycerol phenylbutyrate to a patient in need thereof, wherein said patient is also being treated with midazolam or a pharmaceutically acceptable salt thereof, comprising administering to the patient a therapeutically effective amount of the glycerol phenylbutyrate, and monitoring the therapeutic effect of the midazolam or a pharmaceutically acceptable salt thereof.

These and other embodiments of the disclosure are described in detail below.

As used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the disclosure pertains. Specific terminology of particular importance to the description of the present disclosure is defined below.

As used herein, “adjusting administration”, “altering administration”, “adjusting dosing”, or “altering dosing ” are all equivalent and mean tapering off, reducing or increasing the dose of the substance, ceasing to administer the substance to the patient, or substituting a different active agent for the substance. As used herein, “administering to a patient” refers to the process of introducing a composition or dosage form into the patient via an art-recognized means of introduction.

As used herein, a “dose” means the measured quantity of an active agent to be taken at one time by a patient.

As used herein, “dosing regimen” means the dose of an active agent taken at a first time by a patient and the interval (time or symptomatic) at which any subsequent doses of the active agent are taken by the patient. The additional doses of the active agent can be different from the dose taken at the first time.

As used herein, “effective amount” and “therapeutically effective amount” of an agent, compound, drug, composition or combination is an amount which is nontoxic and effective for producing some desired therapeutic effect upon administration to a subject or patient (e.g., a human subject or patient).

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “ammonia levels” refers to a patient's blood plasma ammonia. In some embodiments, the “normal ammonia level” for a patient is a concentration less than 35 μmol/L.

As used herein, the “elevated ammonia levels” refers to refers to a patient's blood plasma ammonia concentration equal to or greater than the patient's normal ammonia level, e.g., 35 μmol/L. In some embodiments, the ULN is normalized to 35 μmol/L in blood plasma.

Ammonia levels, both “normal” and “elevated”, can vary based on testing methodology (e.g., enzymatic versus colorimetric, μmol/L versus μg/mL) and from laboratory to laboratory. Two units, μmol/L and μg/dL, can be used for the ammonia data. The conversion formula is μg/dL×0.5872=μmol/L. Ammonia values from different labs can be normalized to 9-35 μmol/L. However, the standard normal reference range to be used for patients 2 months of age to less than 2 years of age is 28-57 μmol/L). Normalization can be done by applying the scale normalization approach using the following formula:

s=x*(U_{S}/U_{X}),

where s is the normalized laboratory value, x is the original laboratory value, U_{X} is the ULN reference range from the original laboratory, and U_{S} is the ULN of the normal reference range for the standard laboratory. For example, if a value of 10 was obtained from a local laboratory with a normal range of 5 to 25, and one wishes to normalize this value to the standard reference range which was established to be 28 to 57, then by applying the above formula, a normalized value of 23 would be obtained, accordingly:

s=10*(57/25)=23

Collection and measurement of a patient's blood plasma ammonia levels are known to those of skill in the art. Notably, fasting blood plasma ammonia levels demonstrate the least variability and offer a practical means for predicting the risk and frequency of an HA crisis. In some embodiments, the patient's blood plasma ammonia levels are assayed after fasting. In some embodiments, a patient's blood plasma ammonia level is assayed using venous blood samples. However, for the purposes of this disclosure, additional, standardized methods of blood plasma ammonia collection and measurement, such as by finger prick, may also be suitable.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

“Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “subject” or “individual” or “patient” refers to any patient for whom or which therapy is desired, and generally refers to the recipient of the therapy.

As used herein, “a substance having a narrow therapeutic index” means a substance falling within any definition of narrow therapeutic index as promulgated by the U.S. Food and Drug Administration or any successor agency thereof, for example, a substance having a less than 2-fold difference in median lethal dose (LD50) and median effective dose (ED50) values or having a less than 2-fold difference in the minimum toxic concentration and minimum effective concentration in the blood; and for which safe and effective use of the substance requires careful titration and patient monitoring.

As used herein, a substance is a “substrate” of enzyme activity when it can be chemically transformed by action of the enzyme on the substance. “Enzyme activity” refers broadly to the specific activity of the enzyme (i.e., the rate at which the enzyme transforms a substrate per mg or mole of enzyme) as well as the metabolic effect of such transformations. Thus, a substance is an “inhibitor” of enzyme activity when the specific activity or the metabolic effect of the specific activity of the enzyme can be decreased by the presence of the substance, without reference to the precise mechanism of such decrease. For example a substance can be an inhibitor of enzyme activity by competitive, non-competitive, allosteric or other type of enzyme inhibition, by decreasing expression of the enzyme, or other direct or indirect mechanisms. Similarly, a substance is an “inducer” of enzyme activity when the specific activity or the metabolic effect of the specific activity of the enzyme can be increased by the presence of the substance, without reference to the precise mechanism of such increase. For example, a substance can be an inducer of enzyme activity by increasing reaction rate, by increasing expression of the enzyme, by allosteric activation or other direct or indirect mechanisms. Any of these effects on enzyme activity can occur at a given concentration of active agent in a single sample, donor, or patient without regard to clinical significance. It is possible for a substance to be a substrate, inhibitor, or inducer of an enzyme activity. For example, the substance can be an inhibitor of enzyme activity by one mechanism and an inducer of enzyme activity by another mechanism. The function (substrate, inhibitor, or inducer) of the substance with respect to activity of an enzyme can depend on environmental conditions. Lists of inhibitors, inducers and substrates for CYP3A4 can be found, for instance, at http://www.genemedrx.com/Cytochrome_P450_Metabolism_Table.php, and other sites.

As used herein, “treating” or “treatment” refers to reduction in severity and/or frequency of symptoms, elimination of symptoms and/or underlying cause, and improvement or remediation of damage. In some aspects, the term “treating” and “treatment” as used herein refer to the prevention of the occurrence of symptoms. In some aspects, the term “treating” and “treatment” as used herein refer to the prevention of the underlying cause of symptoms associated with obesity, excess weight, and/or a related condition.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Provided is a method of administering glycerol phenylbutyrate to a patient in need thereof, wherein said patient is also being treated with a CYP2C9 substrate wherein the CYP2C9 substrate is celecoxib, comprising administering to the patient a therapeutically effective amount of the glycerol phenylbutyrate.

In some embodiments, CYP2C9 is human CYP2C9 (Entrez Gene ID: 1559; reference protein sequence Genbank NP_000762), and includes any CYP2C9 allelic variants. In some embodiments, CYP2C9 includes any allelic variants included in the list of human CYP2C9 allelic variants maintained by the Human Cytochrome P450 (CYP) Allele Nomenclature Committee. In some embodiments, it includes any of the *1 through *24 alleles. Additional reference amino acid sequences for human CYP2C9 include Genbank CAH71303, AAP88931, AAT94065, AAW83816, AAD13466, AAD13467, AAH20754, AAH70317, BAA00123, AAA52159, AAB23864, P11712, Q5EDC5, Q5VX92, Q6IRV8, Q8WW80, Q9UEH3, and Q9UQ59.

Also provided is a method of administering glycerol phenylbutyrate to a patient in need thereof, wherein the patient is also being treated with a CYP3A4 substrate having a narrow therapeutic index, comprising administering to the patient a therapeutically effective amount of the glycerol phenylbutyrate, and monitoring the therapeutic effect of the CYP3A4 substrate.

Also provided is a method of administering glycerol phenylbutyrate to a patient in need thereof, comprising administering to the patient a therapeutically effective amount of the glycerol phenylbutyrate; determining that a CYP3A4 substrate having a narrow therapeutic index is also being administered to the patient; monitoring the therapeutic effect of the CYP3A4 substrate.

In some embodiments, monitoring the therapeutic effect of the CYP3A4 substrate comprises determining whether the patient experiences an adverse reaction associated with decreased CYP3A4 substrate plasma concentration.

In one embodiment, the method comprises determining for a patient to whom glycerol phenylbutyrate is going to be administered or is being administered whether a substance that is currently being or will be administered to the patient is a CYP3A4 substrate having a narrow therapeutic index; and determining risk for the patient of an adverse event during coadministration of glycerol phenylbutyrate and CYP3A4 substrate having a narrow therapeutic index.

In some embodiments, the method further comprises informing the patient that the efficacy of the CYP3A4 substrate may be reduced.

In some embodiments, the method further comprises administering an increased dosage of the CYP3A4 substrate.

In some embodiments, the CYP3A4 substrate is chosen from alfentanil, astemizole, cisapride, cyclosporine, diergotamine, ergotamine, fentanyl, irinotecan, pimozide, quinidine, sirolimus, tacrolimus, and terfenadine.

In some embodiments, the CYP3A4 substrate is chosen from alfentanil, quinidine, and cyclosporine.

Also provided is a method of administering glycerol phenylbutyrate to a patient in need thereof, wherein said patient is also being treated with a CYP3A4 substrate wherein the CYP3A4 substrate is midazolam or a pharmaceutically acceptable salt thereof, comprising administering to the patient a therapeutically effective amount of the glycerol phenylbutyrate, and monitoring the therapeutic effect of the midazolam or a pharmaceutically acceptable salt thereof.

Also provided is a method of administering glycerol phenylbutyrate to a patient in need thereof, comprising administering to the patient a therapeutically effective amount of the glycerol phenylbutyrate; determining that midazolam or a pharmaceutically acceptable salt thereof is also being administered to the patient; monitoring the therapeutic effect of the midazolam or a pharmaceutically acceptable salt thereof.

In some embodiments, monitoring the therapeutic effect of the midazolam or a pharmaceutically acceptable salt thereof comprises determining whether the patient experiences an adverse reaction associated with decreased midazolam or a pharmaceutically acceptable salt thereof plasma concentration.

In one embodiment, the method comprises determining for a patient to whom glycerol phenylbutyrate is going to be administered or is being administered whether a substance that is currently being or will be administered to the patient is midazolam or a pharmaceutically acceptable salt thereof; and determining risk for the patient of an adverse event during coadministration of glycerol phenylbutyrate and midazolam or a pharmaceutically acceptable salt thereof.

In some embodiments, the method further comprises informing the patient that the efficacy of the midazolam or a pharmaceutically acceptable salt thereof may be reduced.

In some embodiments, the method further comprises administering an increased dosage of the midazolam or a pharmaceutically acceptable salt thereof.

In some embodiments, CYP3A4 is human CYP3A4 (Entrez Gene ID: 1576; reference protein sequence Genbank NP_059488), and includes any CYP3A4 allelic variants. Specifically, CYP3A4 includes any allelic variants included in the list of human CYP3A4 allelic variants maintained by the Human Cytochrome P450 (CYP) Allele Nomenclature Committee; more specifically it includes any of the *1 through *20 alleles. Additional reference amino acid sequences for human CYP3A4 include Genbank AAF21034, AAG32290, AAG53948, EAL23866, AAF13598, CAD91343, CAD91645, CAD91345, AAH69418, AAI01632, BAA00001, AAA35747, AAA35742, AAA35744, AAA35745, CAA30944, P05184, P08684, Q6GRKO, Q7Z448, Q86SK2, Q86SK3, and Q9BZM0.

In some embodiments, the patient is 2 years of age or older.

In some embodiments, the glycerol phenylbutyrate is administered daily in three equally divided dosages.

In some embodiments, the patient is 2 years of age or older and the glycerol phenylbutyrate is administered daily in three equally divided dosages.

In some embodiments, the patient is between 2 months of age to less than 2 years of age.

In some embodiments, the glycerol phenylbutyrate is administered daily in three or more equally divided dosages.

In some embodiments, the patient is between 2 months of age to less than 2 years of age and the glycerol phenylbutyrate is administered daily in three or more equally divided dosages.

In some embodiments, the method further comprises restricting the patient's dietary protein.

In some embodiments, the therapeutically effective amount of the glycerol phenylbutyrate is 4.5 to 11.2 mL/m²/day (5 to 12.4 g/m²/day).

In some embodiments, the method further comprises monitoring the patient's plasma ammonia levels to determine the need for dosage titration of the glycerol phenylbutyrate.

In some embodiments, the patient is 6 years and older with an elevated plasma ammonia and the method further comprises increasing the glycerol phenylbutyrate dosage to reduce the fasting ammonia level to less than half the upper limit of normal.

In some embodiments, the patient is an infant or pediatric and the method further comprises adjusting the glycerol phenylbutyrate dosage to keep the first ammonia of the morning below the upper limit of normal.

In some embodiments, the method further comprises obtaining measurements of plasma phenylacetate (PAA) concentrations and the ratio of plasma PAA to phenylacetylglutamine (PAGN).

In some embodiments, the method further comprises obtaining measurements of urinary phenylacetylglutamine (U-PAGN). In some embodiments, if the U-PAGN excretion is insufficient to cover daily dietary protein intake and/or the fasting ammonia is greater than half the upper limit of normal, the method further comprises increasing the glycerol phenylbutyrate dosage.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

Effects of PBA and PAA on the CYP enzymes were studied using cultured human hepatocytes for the induction of CYP1A2 and CYP3A4 and using human liver microsomes for the inhibition of CYP1A2, CYP2C8, CYP2C9, CYP2C19, CYP2D6 and CYP3A4/5.

In vitro studies suggest that it is unlikely that PBA and PAA induce CYP1A2 and CYP3A4 in vivo. The levels of induction of CYP1A2 and CYP3A4/5 after treatment with up to 8.6 mM PBA or up to 20.7 mM PAA were low compared to those of the positive controls (omeprazole or rifampicin, respectively) (see below). There was minimal induction (<1.6-fold) of CYP1A2 by either PBA or PAA in cultured human hepatocytes. A control inducer omeprazole at 100 mM induced CYP1A2 activity by 4-9 fold.

TABLE 1
Effect of PBA and PAA treatment on CYP1A2 induction

| | | | Relative | Omeprazole |
|---|---|---|---|---|
| Heptatocyte | PBA | Fold | potency | (fold |
| donor | (mM)* | induction | (%) | induction) |
| Hu0999 8. | 6 | 1.1 | 1.8 | 8.7 |
| Hu4156 2. | 87 | 1.2 | 4.3 | 4.6 |
| Hu4199 2. | 87 | 1.2 | 2.1 | 8.8 |
| | | | Relative | Omeprazole |
| Heptatocyte | PAA | Fold | potency | (fold |
| donor | (mM)* | induction | (%) | induction) |
| Hu0999 20 | 0.7 | 1.3 | 5.8 | 6.3 |
| Hu4156 20 | 0.7 | 1.2 | 5.8 | 4.5 |
| Hu4199 20 | 0.7 | 1.5 | 6.6 | 9.0 |

*Concentration at which maximum fold induction and relative potency occured

There was minimal induction of CYP3A4/5 by PBA in cultured human hepatocytes from 2 of the 3 donors under the conditions of this study. In the third donor there was >2-fold induction of CYP3A4/5 by PBA (at concentrations of 2.87 and 8.6 mM). However, the induction of CYP3A4/5 by PBA in this donor was not concentration-dependent and the extent of induction was about 50% lower compared to the positive control (rifampicin).

There was minimal induction (<1.8-fold) of CYP3A4/5 by PAA in cultured human hepatocytes under the conditions of this study. The effects were not concentration-dependent in every case and there was inter-individual variability in response.

TABLE 2
Effect of PBA and PAA treatment on CYP3A4/5 induction

| | | | Relative | Rifampicin |
|---|---|---|---|---|
| Heptatocyte | PBA | Fold | potency | (fold |
| donor | (mM)* | induction | (%) | induction) |
| Hu0999 2. | 87 | 1.3 | 5.8 | 5.6 |
| Hu4156 8. | 6 | 1.2 | 2.1 | 9.1 |
| Hu0793 2. | 87 | 2.3 | 30.8 | 5.1 |
| | | | Relative | Rifampicin |
| Heptatocyte | PAA | Fold | potency | (fold |
| donor | (mM)* | induction | (%) | induction) |
| Hu0999 6. | 9 | 1.6 | 11.1 | 6.5 |
| Hu4156 20 | 0.7 | 1.7 | 18.2 | 5.1 |
| Hu0793 20 | 0.7 | 1.4 | 13.1 | 4.3 |

On the other hand, in vitro studies suggest that PBA is a reversible inhibitor of CYP2C9, CYP2D6 and CYP3A4/5 while PBA (5 mM (0.821 mg/ml)) did not inhibit CYP1A2, CYP2C8, CYP2C19 or CYP3A4/5 (midazolam 1′-hydroxylase). The inhibition constant, Ki calculated for CYP2C9 and CYP2D6 was 1.3 mM and 1.5 mM, respectively (approximately 0.2 mg/ml for both) and calculation of [J]/Ki ratios were greater than 0.1 suggesting a ‘possible’ in vivo interaction of PBA with CYP2C9 and CYP2D6.

For the inhibition of CYP3A4/5, IC50 was calculated for PBA instead of Ki because of allosteric kinetics characteristics of the reversible inhibition of CYP3A4/5 (testosterone 6-hydroxylase activity). Calculation of [I]/IC50 ratio was greater than 0.1 at all testosterone concentrations suggesting a ‘possible’ in vivo interaction of PBA with CYP3A.

PAA inhibited CYP1A2, CYP2C8, CYP2C9, CYP2C19, CYP2D6 and CYP3A4/5 at 20.7 mM. Based on the initial study result, Ki was further calculated for a representative CYP enzyme, i.e. CYP2C9. The inhibitor constant, Ki calculated for CYP2C9 was 15.1 mM (approximately 2.056 mg/ml) and calculation of [I]/Ki ratio was <0.1 based on mean peak PAA concentration in UCD patients. The [I]/Ki ratio was 0.185 in Cirrhotic-HE patients at 9 ml BID.

TABLE 3
In vitro CYP inhibition study

| | | % Inhibition | | | | | | | |
|---|---|---|---|---|---|---|---|---|---|
| | | | HPN-100 | | PBA | | | PAA (20.7 | |
| | | | (5 mM, | | (5 mM, | | | mM, | |
| | | | 26.5 | | 0.821 | | | 2.818 | |
| | | | mg/ml) | | mg/ml) | | | mg/ml) | |
| | | | Pre- | | Pre- | | | Pre- | |
| | | | incubation | | incubation | | | incubation | |
| | | | time | | time | | | time | |
| | | Selective | (min) | | (min) | | Selective | (min) | |
| CYP | Activity | Inhibitor ¹ | 0 | 30 | 0 | 30 | Inhibitor ² | 0 | 30 |
| CYP1A2 | 7- | 87 | 5 | 8 | 6 | 2 | 87 | 58 | 47 |
| | ethoxyresorufin- | | | | | | | | |
| | O-deethylase | | | | | | | | |
| CYP2C8 | Taxol 6α- | 35 | 11 | 10 | 24 | 27 | 39 | 50 | 49 |
| | hydroxylase | | | | | | | | |
| CYP2C9 | Diclofenac 4′- | 96 | 35 | 19 | 68 | 72 | 97 | 47 | 44 |
| | hydroxylase | | | | | | | | |
| CYP2C19 | S-Mephenytoin | 79 | 8 | 7 | 26 | 19 | 72 | 37 | 37 |
| | 4′-hydroxylase | | | | | | | | |
| CYP2D6 | Bufuralol 1′- | 61 | 1 | 4 | 64 | 63 | 73 | 49 | 57 |
| | hydroxylase | | | | | | | | |
| CYP3A4/ | Testosterone | 98 | 15 | 6 | 80 | 85 | 96 | 60 | 63 |
| 5 | 6β-hydroxylase | | | | | | | | |
| CYP3A4/ | Midazolam 1′- | 97 | 19 | 24 | 2 | −13 | 97 | 63 | 65 |
| 5 | hydroxylase | | | | | | | | |

¹ For HPN-100 and PBA incubations
² For PAA incubations

TABLE 4
[I]/Ki and [I]/IC₅₀ ratios for CYP2C9, CYP2D6,
and CYP3A4/5 in different patient populations - PBA

| | | | | [I]/IC₅₀³ |
|---|---|---|---|---|
| | PBA | [I]/Ki | [I]/Ki | CYP3A4/5 |
| | Mean peak | CYP2C9 | CYP2D6 | IC₅₀ |
| | concentration | (Ki = 0.212 | (Ki = 0.243 | (0.297-0.535 |
| | (mg/ml) | mg/ml) | mg/ml) | mg/ml) |
| UCD | 0.0956 | 0.451 | 0.393 | 0.325-0.179 |
| pediatric | | | | |
| UCD adult | 0.0701 | 0.331 | 0.288 | 0.238-0.131 |
| Healthy | 0.037 | 0.175 | 0.152 | 0.126-0.069 |
| volunteer | | | | |
| Cirrhotic - | 0.1412 | 0.666 | 0.581 | 0.480-0.264 |
| HE 9 | | | | |
| ml BID | | | | |

³The inhibitory effect of PBA on CYP3A4/5 showed an allosteric inhibition; therefore, a calculation of Ki was not possible. Instead IC50 values were calculated.

TABLE 5
[I]/Ki ratios for CYP2C9 in different patient populations - PAA

| | PAA | [I]/Ki |
|---|---|---|
| | Mean peak | CYP2C9 |
| | concentration | (Ki = 2.056 |
| | (mg/ml) | mg/ml) |
| UCD pediatric | 90.5 | 0.044 |
| UCD adult | 40.5 | 0.0197 |
| Healthy volunteer | 25.5 | 0.0072 |
| Cirrhotic - HE 9 ml BID | 381.35 (1.9- | 0.185 (0.0009- |
| | 652.3) | 0.317) |

### Example 2

### An Open Label, Monosequence Crossover Interaction Study to Evaluate the Effect of Steady-State RAVICTI® (Glycerol Phenylbutyrate) Oral Liquid on Cytochrome P450 3A4 Activity Measured by the Pharmacokinetics of Midazolam in Healthy Adult Subjects

### Objectives:

Primary: To examine the effect of steady-state RAVICTI® metabolites on the single-dose pharmacokinetics (PK) of midazolam in healthy subjects.

Secondary: To determine the safety and tolerability of the coadministration of RAVICTI® with midazolam in healthy subjects.

Methodology: This was a Phase 1, open-label, monosequence crossover, drug-drug interaction study of RAVICTI® and midazolam in healthy male and female subjects.

Number of Subjects (Planned and Analyzed): A total of 24 subjects were enrolled in the study and 24 subjects completed the study. All subjects were included in the PK and safety analyses.

Diagnosis and Main Criteria for Inclusion: All subjects enrolled in this study were judged by the Investigator to be normal, healthy volunteers who met all inclusion and none of the exclusion criteria.

Test Product, Dose, Duration and Mode of Administration: Each dose of RAVICTI® (glycerol phenylbutyrate) Oral Liquid, which contains 1.1 g/mL (1.02 g/mL PBA), was administered orally just prior to administration of a standard meal. Single doses of midazolam HCl syrup (2 mg/mL) were administered in the morning following an approximate 10-hour fast on Days 1 and 5 (coadministered with RAVICTI® on Day 5) just prior to administration of breakfast (˜5 minutes before breakfast).

Duration of Treatment: Length of Confinement: approximately 24 hours prior to the first dose of midazolam until approximately 24 hours after the last administration of midazolam (7 days). A follow-up visit took place 6 to 8 days after the last dose of the study drug. Planned Study Conduct Duration: 2 weeks

### Criteria for Evaluation:

Pharmacokinetics: Blood samples for the analysis of plasma midazolam and its metabolite (1′-OH-midazolam, free+conjugates) levels were collected via an indwelling catheter and/or via direct venipuncture. Blood samples were collected on Days 1 and 5 at the following time points: predose, 0.5, 1, 2, 3, 4, 6, 8, 10, 12, 16, and 24 hours postdose. In addition, five plasma PK samples for GPB analysis were collected on Day 4 at the following time points: at 1 minute, 30 minutes, and 1 hour following the first morning dose of RAVICTI; at 30 minutes and 1 hour post second dose of RAVICTI. PK parameters for midazolam and 1′-OH-midazolam in plasma were computed following Day 1 and Day 5 blood draws and included AUC0-t, AUC0-∞, AUCextr (%), Cmax, tmax, kz, λ1/2, and CL/F. Five plasma samples for the analysis of glycerol phenylbutyrate were collected on Day 4 to determine whether complete hydrolysis of glycerol phenylbutyrate occurs in humans.

Safety: Safety and tolerability were assessed through adverse events (AEs), clinical laboratory results, physical examination findings, vital sign measurements, and electrocardiograms (ECGs).

### Statistical Methods:

Pharmacokinetics: The possible drug-drug interaction was examined between coadministration of RAVICTI and midazolam (test) and midazolam administered alone (reference). An analysis of variance (ANOVA), with treatment as a fixed effect and subject as a random effect, was performed. Data for Cmax, AUC0-t, and AUC0-∞, as appropriate, were natural log (ln)-transformed prior to analysis. The 90% confidence intervals (CIs) of the test group means relative to the reference group means were obtained by taking the antilog of the corresponding 90% CIs for the differences between the means on the log scale, i.e., ratio of geometric least-squares (LS) means. It was to be concluded that no drug-drug interaction between RAVICTI (phenylbutyrate [PBA] and phenylacetate [PAA]) and midazolam exists if the antilog of the 90% CIs from the lntransformed Cmax, AUC0-t, and AUC0-∞ (for midazolam and 1′-OH midazolam), as appropriate (provided that the terminal elimination phase was well defined for both analytes), were entirely contained within the interval of 80% to 125%. If the antilog of the 90% CIs of Cmax, AUC0-t, and AUC0-∞, as appropriate, were not contained within the interval of 80% to 125%, it was to be concluded that a drug-drug interaction between RAVICTI (PBA and PAA) and midazolam exists.

Safety: Safety data were summarized by treatment and time point. Descriptive statistics (mean, standard deviation [SD], minimum, median, maximum, and sample size [N]) were calculated for quantitative safety data and frequency counts were compiled for classification of qualitative safety data. AE verbatim terms were mapped to preferred terms and system organ classes using the Medical Dictionary for Regulatory Activities (MedDRA®) (Version 16.1). Concomitant medications were coded with the World Health Organization (WHO) Dictionary version 1Mar. 2013.

Pharmacokinetic Results: The statistical comparisons of plasma midazolam and 1′-OH-midazolam PK parameters are summarized in the following tables.

TABLE 6
Statistical Comparisons of Plasma Midazolam Pharmacokinetic
Parameters Following Administration of Midazolam Alone (Day
1) and When Coadministered With RAVICTI ® (Day 5)

| | Geometric LS Means | | | |
|---|---|---|---|---|
| | Midazolam | Midazolam + | | Confidence |
| | Alone | RAVICTI ® | | Intervals |
| | (Reference) | (Test) | % Mean | 90% |
| Parameter | Day 1 | Day 5 | Ratio | Confidence |
| AUC0-∞ | 66.958 | 46.210 | 69.01 | 65.29-72.95 |
| (ng*hr/mL) | | | | |
| AUC0-t | 64.668 | 44.121 | 68.23 | 64.64-72.01 |
| (ng*hr/mL) | | | | |
| Cmax (ng/mL) | 16.776 | 12.459 | 74.27 | 66.50-82.93 |

Parameters were ln-transformed prior to analysis.
Geometric least-square means (LS Means) are calculated by exponentiating the LS Means from the ANOVA.
% Mean Ratio = 100*(Test/Reference)
Midazolam Alone: 3 mg midazolam single dose (Day 1)
Midazolam + RAVICTI ®: 4.4 g RAVICTI ® TID + 3 mg midazolam single dose (Day 5)

TABLE 7
Statistical Comparisons of Plasma 1′-OH-midazolam Pharmacokinetic
Parameters Following Administration of Midazolam Alone (Day
1) and When Coadministered With RAVICTI ® (Day 5)

| | Geometric LS Means | | | |
|---|---|---|---|---|
| | Midazolam | Midazolam + | | Confidence |
| | Alone | RAVICTI ® | | Intervals |
| | (Reference) | (Test) | % Mean | 90% |
| Parameter | Day 1 | Day 5 | Ratio | Confidence |
| AUC0-∞ | 221.189 | 353.503 | 159.82 | 149.94-170.35 |
| (ng*hr/mL) | | | | |
| AUC0-t | 205.437 | 325.737 | 158.56 | 149.90-167.71 |
| (ng*hr/mL) | | | | |
| Cmax (ng/mL) | 55.366 | 70.646 | 127.60 | 113.80-143.07 |

Parameters were ln-transformed prior to analysis.
Geometric LS Means are calculated by exponentiating the LS Means from the ANOVA.
% Mean Ratio = 100*(Test/Reference)
Midazolam Alone: 3 mg midazolam single dose (Day 1)
Midazolam + RAVICTI ®: 4.4 g RAVICTI ® TID + 3 mg midazolam single dose (Day 5)

Statistical analysis of Cmax, AUC0-t, and AUC0-∞ confirmed a significant drug-drug interaction between RAVICTI metabolites and midazolam. Midazolam peak and overall exposure was reduced upon coadministration with multiple-dose RAVICTI, by approximately 26% and 32%, respectively. The 90% CIs of the mean ratios did not fall within the 80 to 125% target range, nor did they contain 100%.

Statistical analysis of Cmax, AUC0-t, and AUC0-inf confirmed a significant drug-drug interaction between RAVICTI metabolites and midazolam's metabolite, 1′-OH-midazolam. Overall, total 1′-OH-midazolam exposure (based on AUC) was increased upon coadministration with multiple-dose RAVICTI, by approximately 60%. Cmax was increased by 28% with coadministration of treatments. The 90% CIs of the mean ratios did not fall within the 80 to 125% target range, nor did they contain 100%.

Plasma glycerol phenylbutyrate concentrations were not quantifiable (LLOQ=1.00 ng/mL) in any of the Day 4 samples indicating complete hydrolysis of glycerol phenylbutyrate in humans.

Safety Results: There were no deaths, serious adverse events (SAEs), or subject discontinuations due to AEs in this study. Overall, a total of 10 TEAEs were experienced by 6 subjects in this study. One (1) subject experienced 2 laboratory (urinalysis) AEs, considered possibly related to RAVICTI. Additionally, the PI considered 1 episode each of headache, nausea, and flatulence to be possibly/probably related to RAVICTI and 2 episodes of lower abdominal pain to be possibly related to midazolam. There were no clinically significant trends noted in AE, laboratory, vital sign, ECG, or physical examination assessments in this study with respect to subject safety.

Conclusions: Oral doses of RAVICTI coadministered with midazolam appeared to be safe and generally well tolerated in this group of healthy adult male and female subjects. Intact glycerol phenylbutyrate was not detectable in plasma in this study, indicating complete intestinal hydrolysis of glycerol phenylbutyrate in humans.

Steady-state RAVICTI metabolites interacted with the single-dose pharmacokinetics of midazolam and its 1′-hydroxy metabolite; peak and overall midazolam exposure was reduced by 26% and 32%, respectively, and overall 1′-hydroxy-midazolam (free+conjugates) exposure was increased by 60%, while peak exposure increased by 28%. Therefore, RAVICTI may be a weak inducer of CYP3A4 enzyme and coadministration of RAVICTI with drugs that are metabolized by CYP3A4 may result in lower plasma concentrations and/or effect of these drugs.

In healthy subjects, when oral midazolam was administered after multiple doses of RAVICTI (4 mL three times a day for 3 days) under fed conditions, the mean Cₘₐₓ and AUC for midazolam were 25% and 32% lower, respectively, compared to administration of midazolam alone. In addition the mean Cₘₐₓ and AUC for 1-hydroxy midazolam were 28% and 58% higher, respectively, compared to administration of midazolam alone

### Example 3

### An Open-Label, Monosequence Crossover Interaction Study to Evaluate the Effect of Steady-State RAVICTI® (Glycerol Phenylbutyrate) Oral Liquid on Cytochrome P450 2C9 Activity Measured by the Pharmacokinetics of Celecoxib in HealthyAdult Subjects

### Objectives:

Primary: To examine the effect of steady-state dosing of RAVICTI and its metabolites on the single-dose pharmacokinetics (PK) of celecoxib in healthy adult subjects.

Secondary: To determine the safety and tolerability of the co-administration of RAVICTI with celecoxib in healthy adult subjects.

Methodology: This was an open-label, 2-period, monosequence crossover, drug-drug interaction (DDI) study of RAVICTI and celecoxib in healthy male and female subjects.

Number of Subjects (Planned and Analyzed): A total of 28 subjects were enrolled in the study, and 28 subjects completed the study. All subjects were included in the PK and safety analyses.

Diagnosis and Main Criteria for Inclusion: All subjects enrolled in this study were judged by the Principal Investigator (PI) to be normal, healthy volunteers who met all inclusion and none of the exclusion criteria.

Test Product, Dose, Duration, and Mode of Administration: In Treatment A (Period 1), subjects received 200 mg celecoxib (1×200 mg capsule) administered orally with approximately 240 mL of water following an overnight fast and approximately 5 minutes prior to the start of a standard breakfast on Day 1. In Treatment B (Period 2), subjects received 4.4 g RAVICTI (4 mL of 1.1 g/mL glycerol PBA oral liquid) administered orally with approximately 236 mL of water 3 times a day (TID) for 6 consecutive days (Days 1-6), approximately 5 minutes prior to a standard meal, with 200 mg celecoxib (1×200 mg capsule) co-administered on Day 4 with the dosing water immediately after the RAVICTI dose, following an overnight fast and approximately 5 minutes prior to a standard breakfast.

Duration of Treatment: Subjects were housed from the day prior to dosing on Day 1 of Period 1, until after the 72-hour blood draw on Day 7 of Period 2. Subjects returned for follow-up study procedures for approximately 7 days after the last study day of Period 2. There were 2 periods, Period 1 of approximately 4 days and Period 2 of approximately 7 days. The washout phase was 3 days between the celecoxib dose in Period 1 and the first dose of RAVICTI in Period 2.

### Criteria for Evaluation:

Pharmacokinetics: Blood samples (4 mL) for the analysis of plasma celecoxib were collected on Days 1 (Period 1) and 4 (Period 2) at the following time points: predose (Hour 0) and 1, 2, 3, 4, 6, 8, 12, 16, 24, 36, 48, 60, and 72 hours postdose. A blood sample (6 mL) for CYP2C9 genotyping was collected at screening to exclude any subjects with a slow metabolizer genotype (i.e., CYP2C9*2/*2, CYP2C9*2/*3, CYP2C9*1/*3, and CYP2C9*3/*3). PK parameters for celecoxib in plasma were computed following Day 1 (Period 1) and Day 4 (Period 2) blood draws and included AUC0-t, AUC0-inf, AUC%extr, Cmax, tmax, t1/2, kel, and CL/F.

Safety: Safety and tolerability were assessed through adverse events (AEs), clinical laboratory results, physical examination findings, vital sign measurements, and electrocardiograms (ECGs).

### Statistical Methods:

Pharmacokinetics: The possible DDI was examined between co-administration of RAVICTI and celecoxib (Treatment B, test) and celecoxib administered alone (Treatment A, reference). An analysis of variance (ANOVA) was performed with treatment as a fixed effect and subject as a random effect. Data for AUC0-t, AUC0-inf, and Cmax, as appropriate, were ln-transformed prior to analysis. The 90% confidence intervals (CIs) of the test group means relative to the reference group means were obtained by taking the antilog of the corresponding 90% CIs for the differences between the means on the log scale, i.e., ratio of geometric least-squares (LS) means. The absence of a DDI between celecoxib and RAVICTI and its metabolites was concluded if the 90% CIs for plasma celecoxib AUC0-t, AUC0-inf, and Cmax geometric mean ratios (GMRs) (Treatment B/Treatment A) fell within the no-effect boundary of 80-125%a.

Safety: Safety data were summarized by treatment and time point. Descriptive statistics (mean, standard deviation [SD], minimum, median, maximum, and sample size [N]) were calculated for quantitative safety data and frequency counts were compiled for classification of qualitative safety data. AE verbatim terms were mapped to preferred terms and system organ classes using the Medical Dictionary for Regulatory Activities (MedDRA®) (Version 18.0). Concomitant medications were coded with the World Health Organization (WHO) Dictionary version 1Mar. 2015.

Pharmacokinetic Results: The statistical comparisons of plasma celecoxib PK parameters following administration of celecoxib alone and when co-administered with RAVICTI are summarized in the following table.

TABLE 8
Statistical Comparisons of Plasma Celecoxib Pharmacokinetic Parameters
Following Celecoxib Co-administered With RAVICTI (Day 4, Period
2) Versus When Administered Alone (Day 1, Period 1)

| | Geometric LS Means | | | |
|---|---|---|---|---|
| | Celecoxib + | Celecoxib | | |
| | RAVICTI | Alone | | 90% |
| Pharmacokinetic | (Treatment B, | (Treatment A, | GMR | Confidence |
| Parameters | Test) | Reference) | (%) | Intervals |
| AUC0-t | 4774.92 | 5174.01 | 92.29 | 88.48-96.26 |
| (ng*hr/mL) | | | | |
| AUC0-inf | 5007.95 | 5444.03 | 91.99 | 88.42-95.70 |
| (ng*hr/mL) | | | | |
| Cmax (ng/mL) | 545.01 | 620.04 | 87.90 | 81.74-94.53 |

Parameters were ln-transformed prior to analysis.
Geometric least-squares means (LS Means) are calculated by exponentiating the LSM from the ANOVA.
Geometric Mean Ratio (GMR) = 100*(Test/Reference)
Celecoxib Alone: 200 mg celecoxib single dose (Day 1 of Period 1)
Celecoxib + RAVICTI: 4.4 g RAVICTI TID (Days 1-6 of Period 2) + 200 mg celecoxib single dose (Day 4 of Period 2)

The 90% CIs around the GMR derived from the analyses of the ln-transformed primary endpoint PK parameters AUC0-t, AUC0-inf, and Cmax were within the pre-specified no-effect boundary of 80-125%, suggesting that steady-state RAVICTI and its metabolites had no effect on the single-dose PK of celecoxib.

Safety Results: There were no deaths, SAEs, or subject discontinuations due to AEs in this study. Overall, a total of 23 TEAEs were experienced by 8 subjects. The majority of the AEs were mild in severity and considered by the PI to be not drug-related. There were no clinically significant trends noted in AE, laboratory, vital sign, ECG, or physical examination assessments in this study.

Conclusions: The single-dose PK profiles of celecoxib following 200 mg of celecoxib administered alone and 200 mg of celecoxib co-administered with multiple doses of 4.4 g of RAVICTI in healthy subjects were similar.

The 90% CIs around the GMRs of celecoxib primary PK endpoints AUC0-t, AUC0-inf, and Cmax following celecoxib co-administered with RAVICTI relative to celecoxib administered alone were contained within the limits of 80 and 125%, suggesting that steady-state RAVICTI and its metabolites had no effect on the single-dose PK of celecoxib. Multiple doses of RAVICTI did not appear to inhibit CYP2C9 activity in vivo. Oral doses of RAVICTI co-administered with celecoxib appeared to be safe and generally well tolerated in this group of healthy adult male and female subjects.

Concomitant administration of RAVICTI did not significantly affect the pharmacokinetics of celecoxib, a substrate of CYP2C9. When 200 mg of celecoxib was orally administered with RAVICTI after multiple doses of RAVICTI (4 mL three times a day for 6 days) under fed conditions (a standard breakfast was consumed 5 minutes after celecoxib administration), the mean Cₘₐₓ and AUC for celecoxib were 13% and 8% lower than after administration of celecoxib alone.

1-20. (canceled)

21. A method of administering glycerol phenylbutyrate to a patient in need thereof, wherein said patient is also being treated with alfentanil or a pharmaceutically acceptable salt thereof, comprising
administering to the patient a therapeutically effective amount of the glycerol phenylbutyrate,
informing the patient that the efficacy of the alfentanil or a pharmaceutically acceptable salt thereof may be reduced; and
monitoring the therapeutic effect of the alfentanil or a pharmaceutically acceptable salt thereof.

22. The method of claim 21, wherein the patient is 2 years of age or older.

23. The method of claim 22, wherein the glycerol phenylbutyrate is administered daily in three equally divided dosages.

24. The method of claim 21, wherein the patient is between 2 months of age to less than 2 years of age.

25. The method of claim 24, wherein the glycerol phenylbutyrate is administered daily in three or more equally divided dosages.

26. The method of claim 21, further comprising restricting the patient's dietary protein.

27. The method of claim 21, wherein the therapeutically effective amount of the glycerol phenylbutyrate is 4.5 to 11.2 mL/m²/day (5 to 12.4 g/m²/day).

28. The method of claim 21, further comprising monitoring the patient's plasma ammonia levels to determine the need for dosage titration of the glycerol phenylbutyrate.

29. The method of claim 28, wherein the patient is 6 years and older with an elevated plasma ammonia and the method further comprises increasing the glycerol phenylbutyrate dosage to reduce the fasting ammonia level to less than half the upper limit of normal.

30. The method of claim 28, wherein the patient is an infant or pediatric and the method further comprises adjusting the glycerol phenylbutyrate dosage to keep the first ammonia of the morning below the upper limit of normal.

31. The method of claim 21, further comprising obtaining measurements of plasma phenylacetate (PAA) concentrations and the ratio of plasma PAA to phenylacetylglutamine (PAGN).

32. The method of claim 21, further comprising obtaining measurements of urinary phenylacetylglutamine (U-PAGN).

33. The method of claim 32, wherein if the U-PAGN excretion is insufficient to cover daily dietary protein intake and/or the fasting ammonia is greater than half the upper limit of normal, the method further comprises increasing the glycerol phenylbutyrate dosage.

34. The method of claim 21, further comprising administering an increased dosage of the alfentanil or a pharmaceutically acceptable salt thereof.
